(19) United States
(12) Patent Application Publication
(10) Pub. No.: US 2021/0236308 A1
(43) Pub. Date: Aug. 5, 2021

(54) FASTENING DEVICE FOR FASTENING A PROSTHESIS SHAFT TO A PROSTHETIC KNEE JOINT, AND PROSTHETIC KNEE JOINT

(71) Applicant: OTTOBOCK SE & CO. KGAA, Duderstadt (DE)

(72) Inventor: Patrick SCHOLL, Darmstadt (DE)

(73) Assignee: OTTOBOCK SE & CO. KGAA, Duderstadt (DE)

(21) Appl. No.: 17/264,786

(22) PCT Filed: Jul. 10, 2019
(86) PCT No.: PCT/EP2019/068533

§ 371 Date: Jan. 29, 2021

(30) Foreign Application Priority Data
Jul. 30, 2018 (DE) 10 2018 118 363.1

(51) Int. Cl. A61F 2/80; A61F 2/64

(52) U.S. Cl. CPC A61F 2/80; A61F 2/64

(57) ABSTRACT

A fastening device for fastening a prosthesis shaft to a prosthetic knee joint that has a proximal top part and a distal bottom part, which are mounted on one another to pivot about a prosthetic knee joint axis. The fastening device has a distal bracket for attachment to the bottom part or to a lower-leg tube. At least one strut extends in the proximal direction from the holder beyond the prosthetic knee joint axis and on the strut a receptacle is arranged proximally to the prosthetic knee joint axis for pivotally mounting the prosthesis shaft about a shaft pivot access. At least one force transfer element is fastened offset to the to the shaft pivot access at a proximal bearing point to transfer a pivoting movement of the prosthesis shaft. The force transfer element is coupled to the top part at a distal bearing point in such a way that a pivoting movement of the prosthesis shaft about the shaft pivot axis causes a pivoting movement of the top part about the prosthetic knee joint axis.

The invention relates to a fastening device for fastening a prosthesis socket to a prosthetic knee joint, and a prosthetic knee joint having such a fastening device. The prosthetic knee joint has a proximal top part and a distal bottom part, which are mounted on each other pivotably about a prosthetic knee joint axis.

In patients with missing limbs, prosthetic fittings on the lower extremities are needed in order to achieve an approximation to the natural appearance and, if possible, to simulate and replace the function of a natural limb. Patients who are missing a knee joint require a prosthetic knee joint, the latter generally being fastened to a prosthesis socket, which is secured on a thigh stump. A number of systems are known for securing the thigh socket on the stump. A common feature of these systems is that the thigh stump is held in the prosthesis socket, and the connection to the ground is produced via a prosthetic knee joint with a lower-leg tube and a prosthetic foot. The prosthetic knee joint permits a pivotable connection of the lower-leg part in relation to the thigh stump. To achieve a gait pattern that is as natural as possible and an outer appearance that is as natural as possible, the prosthetic knee joint axis is if possible arranged at the height of the natural knee joint axis of the intact and sound leg. Otherwise, a very uneven gait pattern would arise. If the thigh stump is comparatively short, the distance can be compensated by a thigh tube.

DE 10 2009 051 668 A1 relates to a knee joint for a prosthesis, comprising a swing phase controller with a piston coupled to a thigh connector piece and movable relative to a cylinder, wherein the cylinder is coupled to a lower-leg connector piece and is pivotable relative to the lower-leg connector piece about at least one axis. By way of an articulation point remote from the axis, the cylinder is connected to a spring means, which is supported on the lower-leg connector piece.

DE 10 2009 053 128 B4 relates to a prosthetic knee joint with a pyramid adapter for fastening a prosthesis socket. Distally with respect to the pyramid adapter, there is a transverse link in which two transverse bores are arranged. The first transverse bore receives two front connecting levers, and the second transverse bore receives one connecting lever. The connecting lever is mounted in an articulated manner on a so-called inhibitor, which in turn is mounted in an articulated manner on a distal bottom part. At a distance therefrom, the two connecting levers are mounted pivotably on the bottom part. The inhibitor is mounted in an oblong hole in the bottom part against a spring element.

WO 03/092545 A2 relates to a prosthetic knee joint having four axial pins, wherein the four axial pins interconnect a knee head, two connecting plates, a fork and a seat for connection to a lower-leg part, and form a triangular support, wherein the point of application is located at the vertex of the inverted triangle formed by the four axial pins.

A particular set of problems arise in patients who have undergone knee disarticulation. In knee disarticulation, the thigh bone remains substantially undamaged; the distal condyles of the thigh bone are substantially preserved. Such treatment is advantageous for a patient in terms of the pressure applied to the soft tissue in the end region of the stump. A problem with knee disarticulation is that a prosthetic knee joint has to be fitted to the distal end of the prosthesis socket, resulting in an additional lengthening of the thigh, for example by a prosthesis liner, the prosthesis socket, a fastening element on the prosthesis socket for securing to the top part of the prosthetic knee joint, and the top part itself. The distance from the hip joint to the prosthetic knee joint axis is sometimes significantly greater than the distance from the hip joint to the natural knee joint axis. This has disadvantages with regard to the walking behavior and also when sitting, for example in public transport with restricted leg room.

The object of the present invention is therefore to make available a fastening device and a prosthetic knee joint with which these disadvantages can be reduced or avoided.

According to the invention, this object is achieved by a fastening device having the features of the main claim, and by a prosthetic knee joint having the features of the additional independent claim. Advantageous embodiments and developments of the invention are disclosed in the dependent claims, the description and the figures.

The fastening device for fastening a prosthesis socket to a prosthetic knee joint that has a proximal top part and a distal bottom part, which are mounted on each other pivotably about a prosthetic knee joint axis, has a distal holder for securing to the bottom part or to a lower-leg tube connected to the bottom part. At least one strut extends in a proximal direction from the holder beyond the prosthetic knee joint axis. A receptacle is arranged on the strut, proximally with respect to the prosthetic knee joint axis, for the pivotably mounting of the prosthesis socket about a pivot axis. At least one force transmission element, offset with respect to the socket pivot axis, i.e. eccentric to the socket pivot axis, is fastened at a proximal bearing point in order to transmit a pivoting movement of the prosthesis socket.

The force transmission element is coupled to the top part, at a distal bearing point, in such a way that a pivoting movement of the prosthesis socket about the socket pivot axis causes a pivoting movement of the top part of the prosthetic knee joint about the prosthetic knee joint axis. By way of the fastening device, it is possible to use a conventional prosthetic knee joint also for patients with a knee disarticulation or with particularly long thigh stumps. The prosthetic knee joint can be designed as a simple locking knee joint with a monocentric pivot axis, as a multi-link knee joint, as a prosthetic knee joint with a damper device, and in particular as a prosthetic knee joint with electronic control for changing an extension resistance and/or flexion resistance. Design changes to the knee joints are not needed by coupling the prosthesis socket to the prosthetic knee joint via the fastening device. Despite the presence of a very long thigh stump with an additional prosthesis socket, which further increases the effective length of the stump, it is possible to provide a prosthetic fitting with segment lengths that correspond to the segment lengths of a sound leg. In this way, an improved gait behavior and a minimized lengthening of the thigh while sitting are achieved. The strut, which is secured and fixed to the bottom part of the prosthetic knee joint or also to a lower-leg tube, is rigidly connected to the bottom part or the lower-leg tube and extends beyond the prosthetic knee joint axis in the proximal direction. The receptacle for the pivotable mounting of the prosthesis socket on the strut can be formed integrally with the strut. Alternatively, a component or an adapter on which the prosthesis socket is directly or indirectly secured can be fastened to the strut. The prosthesis socket can be fixed directly on the strut or the receptacle, for which purpose corresponding receptacles, pins or bearings are arranged or formed on the prosthesis socket. Alternatively, an intermediate piece, by which the prosthesis socket is coupled to the strut or to the receptacle, can be arranged on the prosthesis socket. A direct attachment of the prosthesis socket to the strut or to the struts reduces the number of components that have to be produced and thus also reduces the weight. If intermediate pieces, components or adapters are provided which can be arranged between the strut and the prosthesis socket, it is easier to adapt an individual prosthesis socket to standardized prosthesis components, in particular to a standardized fastening device.

By means of the force transmission device fastened eccentrically or in an offset manner with respect to the socket prosthesis axis, it is possible that forces or moments applied by a pivoting of the prosthesis socket about the socket prosthesis axis can be continued in the distal direction, such that the top part is pivoted relative to the bottom part, corresponding to a pivoting of the prosthesis socket relative to the strut. The force transmission element has a proximal bearing point and a distal bearing point, wherein the distal bearing point is coupled to the top part. The distal bearing point can either be formed directly on the top part or coupled to the top part via a further component. If a component is provided between the top part of the prosthetic knee joint and the force transmission element, this permits easier adaptation to the individual requirements.

The proximal bearing point can be arranged on the prosthesis socket itself, or on a lever which is pivotable about the socket pivot axis and coupled to the prosthesis socket. If the proximal bearing point of the force transmission device is formed on the prosthesis socket itself, the prosthesis socket forms a part of the multi-link gear, which is formed by the at least one strut, the prosthesis socket and the force transmission device. The distal end of the force transmission device can likewise be coupled pivotably to the top part of the prosthetic knee joint, such that three levers mounted pivotably on each other are present, which are in turn coupled to one another by the prosthetic knee joint. As an alternative to an arrangement of the proximal bearing point directly on the prosthesis socket, it is possible that a lever is formed which is coupled to the prosthesis socket for conjoint rotation therewith and to transmit moment, such that, when the prosthesis socket pivots about the socket pivot axis, the lever is pivoted with it. By the embodiment of a lever, it is possible to design the fastening device as a module that can be prefabricated and that has to be coupled at two points to the existing prosthetic knee joint and at one point to the prosthesis socket. The strut is secured on the bottom part or is fixed rigidly with respect to the bottom part on a prosthesis component arranged distally with respect to the prosthetic knee joint axis. The force transmission element is coupled distally to the top part of the prosthetic knee joint, and the prosthesis socket is coupled pivotably to the force transmission device at the at least one strut in such a way that a pivoting movement about the socket pivot axis causes a shifting of the force transmission element. This facilitates the assembly of the final prosthesis, in particular the replacement of prosthetic knee joints with standardized receiving devices for the fastening device. A standardized receiving device for securing the prosthesis socket can be arranged or formed on the fastening device itself.

A bracket, with a receiving device for securing the prosthesis socket to the bracket, can be mounted on the at least one strut. The receiving device can be designed as part of a pyramid adapter, and the bracket can be designed as a one-arm or two-arm bracket. For reasons of stability, two brackets are preferably provided, namely a medial bracket and a lateral bracket, between which the prosthesis socket is received and mounted. If mounting is not carried out directly on the prosthesis socket, fastening can be effected via a bracket that is mounted pivotably in the receptacle on the strut, so as to compensate for size deviations and to permit adaptation to different sizes of prosthesis socket. In the case of two struts, a two-arm bracket is recommended, so as to permit medial and lateral mounting and a pivoting movement about a common socket pivot axis. A receiving device, for example a customary pyramid adapter component, can be arranged on the bracket, for example centrally between the bracket ends, such that a conventional prosthesis socket with the usual distal attachment means for securing to a prosthetic knee joint can be used to permit coupling and binding of the prosthesis socket to the fastening device and thus to the prosthetic knee joint. The receiving device secures the prosthesis socket on the bracket in a rotationally rigid manner and to transmit moment, such that a movement of the prosthesis socket relative to the bottom part of the prosthetic knee joint, and, on account of the rigid binding of the at least one strut to the bottom part, also a movement of the prosthesis socket relative to the strut via the bracket, leads to a pivoting movement about the socket pivot axis and, on account of the eccentric mounting of the force transmission element and its coupling to the top part, to a relative movement of the top part of the prosthetic knee joint relative to the bottom part of the prosthetic knee joint.

The distal bearing point of the force transmission device can be arranged on a carrier, which has a coupling device for securing to the top part. The coupling device can be designed as part of a pyramid adapter, which is formed or arranged on the carrier. The carrier itself can be designed, for example, as a plate or jib which has a receptacle or several receptacles for securing, in particular for pivotably securing, the force transmission device. By virtue of the pivotable mounting both at the distal bearing point and at the proximal bearing point, it is possible for the rotation movement of the prosthesis socket relative to the strut or struts to be converted into a linear movement, which is in turn converted into a rotation movement of the top part about the prosthetic knee joint axis. The force vector from the proximal bearing point to the distal bearing point is configured such that it is oriented at a distance from the prosthetic knee joint axis over the course of the entire pivoting movement of the prosthesis socket, such that there is no dead center position, and pivoting of the prosthesis socket beyond the desired pivoting range is blocked.

In a development of the invention, provision is made that the force transmission element is designed as a bracket with two branches which are arranged medially and laterally with respect to the prosthesis socket. The brackets form two proximal bearing points and are connected to each other distally via a crosspiece. The crosspiece and the branches are connected to each other pivotably about a pivot axis. The pivot axis of the two branches is at a distance from the pivot axis of the prosthetic knee joint. The force transmission element is mounted pivotably at the proximal bearing point. The force transmission element is preferably also mounted pivotably at the distal bearing point.

The prosthesis socket can have a dimensionally stable end cap on which the proximal bearing points or the at least one proximal bearing point are/is formed. Alternatively or in addition, a distal fastening element for coupling to the strut can be arranged on the prosthesis socket such that, for example, the prosthesis socket can be fastened to an adapter or to a pivotable bracket.

The holder can preferably be secured releasably to the bottom part or to a lower-leg tube. Preferably, the holder is arranged exchangeably on the strut, such that, by exchange of the respective holder, a strut can be coupled to different prosthetic knee joints. The holder secures the strut or the struts rigidly relative to the bottom part.

The prosthesis socket has a distal end which preferably protrudes distally beyond the socket pivot axis. The socket pivot axis is preferably arranged in the region of a natural knee joint axis such that, in relation to the sound leg, identical segment lengths can be achieved. The invention likewise relates to a prosthetic knee joint having a fastening device as described above.

The distance of the proximal bearing point of the force transmission device from the socket pivot axis can be modifiable. For example, several form-fit elements spaced apart from one another, such as bores, threads or bolts, can be arranged on the prosthesis socket or on a lever, in order to change the position of the force transmission device relative to the socket pivot axis. It is likewise possible for the distance of the proximal bearing points and the distal bearing points from one another to be modifiable, for example by means of a plurality of bearing bores spaced apart from one another. It is also possible for the distance of the distal bearing point from the knee joint pivot axis to be adjustable. Instead of the respective distances being modifiable by various fastening elements arranged at fixed distances, it is possible to have a displaceable and fixable arrangement of the bearing points or bearing locations, for example via oblong hole guides and clamping fixing devices.

Latching devices such as teeth can alternatively be provided for stepless displaceability and fixability.

An illustrative embodiment of the invention is explained in more detail below with reference to the accompanying figures, in which:

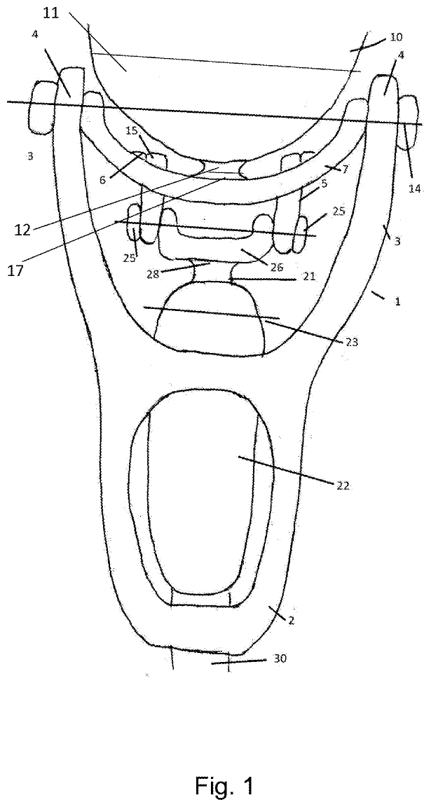

FIG. 1 shows a schematic front view of a prosthetic knee joint with a fastening device;

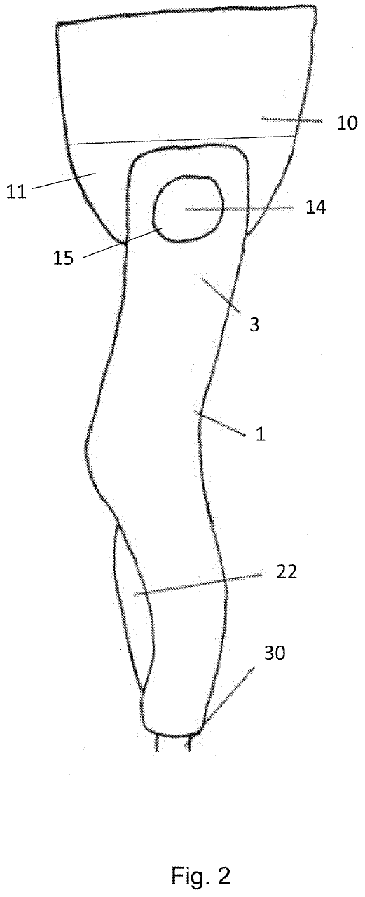

FIG. 2 shows a side view;

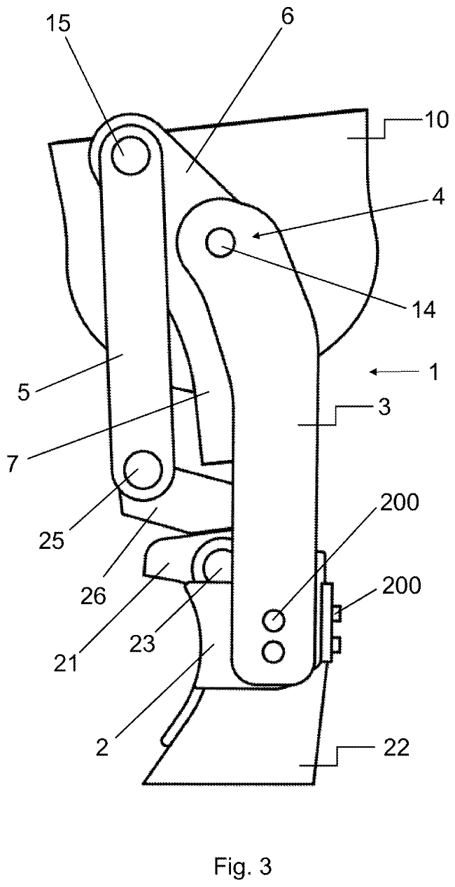

FIG. 3 shows a side view of a variant, in an extended position;

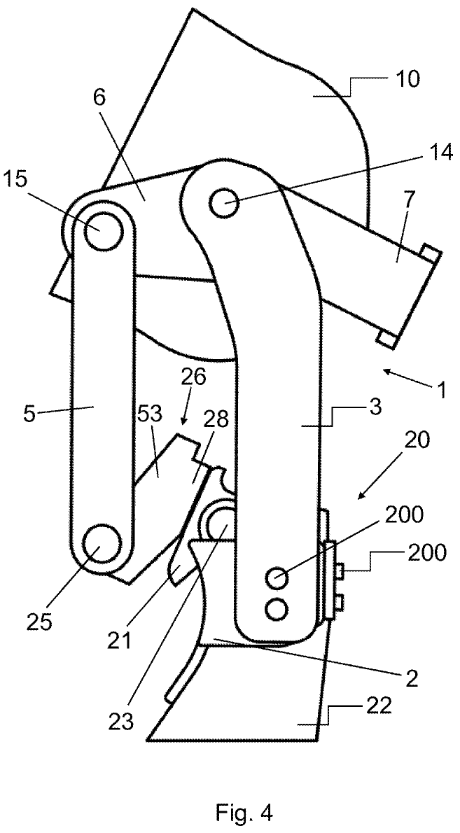

FIG. 4 shows the variant according to FIG. 3 in a flexed position, and

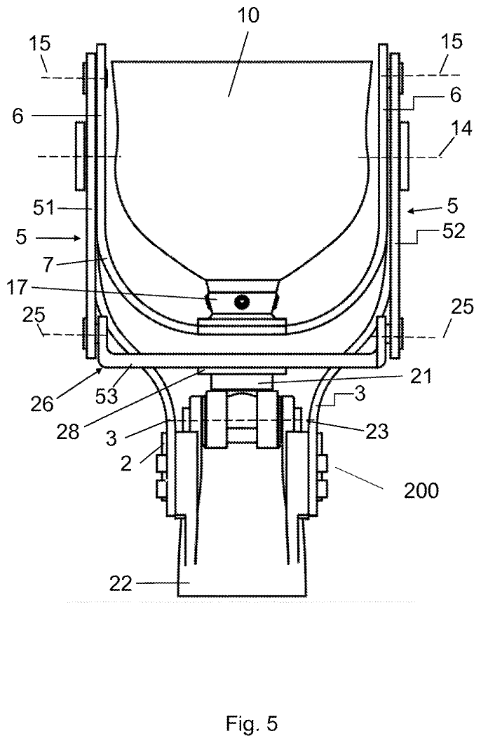

FIG. 5 shows a rear view of the variant according to FIG. 3.

FIG. 1 shows a schematic front view of a prosthetic knee joint 20 having a top part 21 and a bottom part 22, which are connected to each other pivotably about a pivot axis 23. Damper devices, actuators, end stops and control devices and drives for adjusting resistances, stops or the like can be arranged between the top part 21 and the bottom part 22. A lower-leg tube 30, which connects the prosthetic knee joint 20 to a prosthetic foot (not shown), is arranged distally on the bottom part 22. Customary connecting means, for example a pyramid adapter, for securing to a prosthesis socket can be arranged on the top part 21.

On the prosthetic knee joint 20, a fastening device 1 is arranged which, in the distal region, has a holder 2 for securing the fastening device 1 to the bottom part 22 or, as in the illustrative embodiment shown, to the lower-leg tube 30. By way of the holder 2, the fastening device 1 is coupled rigidly to the bottom part 22 or the lower-leg tube 30, for example via form-fit elements or via clamping devices.

The fastening device 1 has two struts 3 or branches 3 which extend from the distal holder 2 in a proximal direction and are arranged medially and laterally with respect to the prosthetic knee joint 20. The two struts 3 or branches 3 are connected to each other, resulting in a U-shaped configuration in the proximal region of the fastening device 1. At the proximal ends of the struts 3 or branches 3, receptacles 4 are formed, for example bearing pins, bearing bores or similar, through which a socket pivot axis 14 runs. Instead of two struts 3 or branches 3, it is possible, in a variant of the invention, to provide only one strut 3 or branch 3, either medially or laterally with respect to the prosthetic knee joint 20. A design with two struts 3 or branches 3 increases the stability and the guiding precision.

A bracket 7 is mounted pivotably about the socket pivot axis 14, which bracket 7 is likewise U-shaped and has its lowest point, i.e. its maximum distal point, at the center between the two struts 3 or branches 3. A receiving device 17, for example a pyramid adapter, is arranged on the bracket 7 and serves to secure a prosthesis socket 10. On the prosthesis socket 10, designed as a dimensionally stable prosthesis socket for receiving a thigh stump or designed with a dimensionally stable end cap 11, in particular for receiving a thigh stump after a knee disarticulation, a distal fastening element 12 is provided, for example a pyramid adapter receptacle, in order to be able to fasten the prosthesis socket 10 to the receiving device 17 reversibly, with form-fit engagement and with the possibility of alignment. By way of the distal fastening element 12 and the receiving device 17, the prosthesis socket 10 is fixed to the bracket 7 for conjoint rotation therewith and to transmit moment. By being secured to the bracket 7, the prosthesis socket 10 can be pivoted about the socket pivot axis 14. The socket pivot axis 14 runs proximally with respect to the distal end of the prosthesis socket 10. Simple exchange of the components is possible by virtue of the reversible securing via the distal fastening element 12 on the receiving device 17.

On the bracket 7 itself, a jib or lever 6 is fastened which is coupled rigidly to the bracket 7. The lever 6 extends rearwardly, i.e. in a posterior direction, and forms a proximal bearing point 15. Two levers 6 are formed on or fastened to the bracket 7 and are arranged symmetrically with respect to the longitudinal extent of the prosthetic knee joint 20. A force transmission element 5 extends in a distal direction from the proximal bearing point 15 on the lever 6, where the respective force transmission element 5 is mounted on a carrier 26 at a distal bearing point 25. The force transmission element 5 is mounted pivotably both at the proximal bearing point 15 and at the distal bearing point 20. The carrier 26 connects the two force transmission elements 5 to each other, such that a further U-shaped bracket is formed. The carrier 26 has a coupling device 28 which corresponds to the upper connecting means of the top part 21, for example a pyramid adapter receptacle, similar to the receiving device on the prosthesis socket 10. When the prosthesis socket 10 pivots about the socket pivot axis 14, and the prosthesis socket 10 and thus the stump perform a flexion relative to the lower-leg tube 30 or the bottom part 22, the bracket 7 and thus the two levers 6 pivot, such that the force transmission devices 5 are moved in a distal direction. On account of the distal movement of the force transmission devices 5, the distal bearing points 25 are subjected to a pressing force. On account of the eccentric arrangement of the bearing points 25 relative to the knee joint pivot axis 23, a moment arises about the prosthetic knee joint axis 23, such that the top part 21 is pivoted relative to the bottom part 22 about the prosthetic knee joint axis 23.

For the prosthesis user, the socket pivot axis 14 thus forms the axis about which the whole prosthetic device pivots, apart from the prosthesis socket 10. By the positioning of the socket pivot axis 14 in the region of the distal end of the prosthesis socket 10, but proximally with respect to the distal end of the prosthesis socket 10, it is possible to position the pivot axis relevant to the user closer to the natural knee joint axis. In this way, it is possible to make available almost identical segment lengths of thigh and lower leg, or of thigh stump and distal prosthesis component. No further change of design of customary prosthetic knee joints 20 is necessary; the connection devices and also the coordination and programming can be kept unchanged. Different leg lengths can be compensated simply by shifting the position of the socket pivot axis 14. Likewise, transmission ratios can be changed by setting and modifying the position of the respective bearing points 15, 25 and the fastening of the prosthesis socket 10 to the struts 3 or branches 3 or the strut 3 or the branch 3.

FIG. 2 shows a side view of the fastening device 1 with the socket pivot axis 24 at the proximal end of the struts 3 or branches 3, the lower-leg tube 30, and the bottom part 22 of the prosthetic knee joint. The prosthesis socket 10 extends in a distal direction beyond the socket pivot axis 14. On the prosthesis socket 10, a dimensionally stable end cap 11 is arranged or formed, via which the prosthesis socket 10 can be secured to the struts 3 or branches 3. The securing can be effected via proximal bearing points 15, such that the end cap 11 can be secured pivotably on the struts 3 or branches 3 either directly or via intermediate pieces.

FIG. 3 shows a schematic sectional view of a variant of the invention, in an extended position. By way of a holder 2 which can be arranged exchangeably on the fastening device 1, said fastening device 1, which is designed as an adapter for securing the prosthesis socket to a conventional prosthetic knee joint 20, is releasably connected with its distal end to the bottom part 22 of the prosthetic knee joint 20 or, in a variant, to the lower-leg tube 30. The struts 3 or branches 3 extend in the proximal direction beyond the distal end of the prosthesis socket 10. The socket pivot axis 14 lies proximally with respect to the distal socket end of the prosthesis socket 10, and the branches of the bracket 7 extend distally with respect to the receiving device 17 (not shown), which serves to receive the prosthesis socket 10. It will be seen from the side view that the levers 6 protrude in a posterior direction, i.e. to the rear in the direction of walking, from the connection site on the receiving device 17, such that a lever arm is formed, with the result that, during a pivoting movement of the prosthesis socket 10 about the socket pivot axis 14, the force transmission element 5, which is mounted pivotably at the proximal bearing point 15 and the distal bearing point 25, is moved downward substantially rectilinearly. The distal bearing point 25 is likewise arranged posteriorly on the carrier 26 in the direction of walking, i.e. behind the knee joint pivot axis 23, such that the force vector from the proximal bearing point 15 to the distal bearing point 25 does not intersect the knee joint pivot axis 23. In this way, a moment is generated about the prosthetic knee joint pivot axis 23, such that the top part 21 is pivoted about the prosthetic knee joint pivot axis 23 relative to the bottom part 22.

The pivoted position is shown in FIG. 4.

The fastening device 1 allows the effective rotation point of the knee to be set to the anatomical height, by way of the socket pivot axis 14. Conventional prosthetic knee joints 20 can also be used for patients with long thigh stumps, in particular for patients on whom knee disarticulation has been carried out. Moment transfer takes place from the fastening device 1 to the conventional prosthetic knee joints. The coupling of the prosthesis socket 10 to the fastening device 1 is effected either via a conventional pyramid adapter or via laminated binding points, by way of which the branches can be connected medially and laterally to the prosthesis socket 10, if appropriate using spacer pieces.

FIG. 5 shows the variant according to FIGS. 3 and 4 in a rear view. It will be seen from FIG. 5 that the prosthesis socket 10, at its distal end, is secured centrally to the bracket 7 via a pyramid adapter as receiving device 17. The embodiment of the receiving device 17 as a pyramid adapter has the advantage that conventional prosthesis sockets 10 can be used which are already configured for direct securing to a prosthetic knee joint. Alternatively, the prosthesis socket 10 can be coupled by other fastening devices to the bracket 7, or by other devices to the one or more force transmission elements 5. In the illustrative embodiment shown, the bracket 7 is continued past the socket pivot axis 14 in the proximal direction, such that the levers 6 form a connection between the socket pivot axis 14 and the bearing points 15. In this way, a force coupling is provided between the prosthesis socket 10 and the proximal bearing points 15, such that, during a pivoting movement of the prosthesis socket 10 about the socket pivot axis 14, the branches 51, 52 of the force transmission element 5 exert a distally directed force on the distal bearing points, on account of the pivotable bearing at the bearing points. The carrier 26, which as a crosspiece connects the medial and lateral branches 51, to each other, is in this way pivoted about the prosthetic knee joint axis 23.

The distal holder is fastened releasably to the bottom part 22 by bolts or screws 200. A damper device, as shown in FIG. 1, is not shown in the variant according to FIGS. 3 to 5. Such a damper device can be fastened to the top part 21 and the bottom part 22. The struts 3 are secured medially and laterally to the bottom part 22 via the screws or bolts 200. Instead of being fastened by form-fit engagement, the struts 3 can also be clamped on the bottom part 22.

1. A fastening device for fastening a prosthesis socket to a prosthetic knee joint that has a proximal top part and a distal bottom part, which are mounted on each other pivotably about a prosthetic knee joint axis, the fastening device comprising:
a distal holder for securing to the bottom part or to a lower-leg tube;
at least one strut extending in a proximal direction from the holder beyond the prosthetic knee joint axis;
a receptacle arranged on the strut, proximally with respect to the prosthetic knee joint axis, for the pivotable mounting of the prosthesis socket about a socket pivot axis;
at least one force transmission element, offset with respect to the socket pivot axis and fastened at a proximal bearing point in order to transmit a pivoting movement of the prosthesis socket, the force transmission element being coupled to the top part, at a distal bearing point, in such a way that a pivoting movement of the prosthesis socket about the socket pivot axis causes a pivoting movement of the top part about the prosthetic knee joint axis.

2. The fastening device according to claim 1, wherein the proximal bearing point is arranged on the prosthesis socket, or on a lever which is pivotable about the socket pivot axis and coupled to the prosthesis socket.

3. The fastening device according to claim 1, further comprising a bracket, with a receiving device for securing the prosthesis socket to the bracket, mounted on the at least one strut.

4. The fastening device according to claim 3, wherein the receiving device is designed as part of a pyramid adapter.

5. The fastening device according to claim 3, wherein the bracket is designed as a one-arm or two-arm bracket.

6. The fastening device according to claim 1, wherein the distal bearing point is arranged on a carrier which has a coupling device for securing to the top part.

7. The fastening device according to claim 6, wherein the coupling device is designed as part of a pyramid adapter.

8. The fastening device according to claim 1, wherein the force transmission element is designed as a bracket with two branches, which are arranged medially and laterally with respect to the prosthesis socket, form two proximal bearing points, and are connected to each other distally via a crosspiece.

9. The fastening device according to claim 1, wherein the force transmission element is mounted pivotably about the proximal bearing point.

10. The fastening device according to claim 1, wherein the prosthesis socket has at least one of a dimensionally stable end cap on which the at least one proximal bearing point is formed or a distal fastening element is arranged for coupling to the strut.

11. The fastening device according to claim 1, wherein the holder can be secured releasably to the bottom part or to a lower-leg tube.

12. The fastening device according to claim 1, wherein the prosthesis socket has a distal end which protrudes distally beyond the socket pivot axis.

13. The fastening device according to claim 12, wherein the socket pivot axis is configured to be arranged in a region of a natural knee joint axis.

14. A prosthetic knee joint having the fastening device according to claim 1.

15. A fastening device to fasten a prosthesis socket to a prosthetic knee joint, the prosthetic knee joint having a proximal top part pivotally mounted to a distal bottom part about a prosthetic knee joint axis, the fastening device comprising:
a distal holder to secure fastening device to the bottom part or to a lower-leg tube;
at least one strut extending in a proximal direction from the holder beyond the prosthetic knee joint axis;
a receptacle arranged on the strut proximally with respect to the prosthetic knee joint axis, the receptacle configured to pivotably mounting the prosthesis socket about a socket pivot axis;
at least one force transmission element offset with respect to the socket pivot axis, fastened at a proximal bearing point, and configured to be coupled to the top part at a distal bearing point in such a way that a pivoting movement of the prosthesis socket about the socket pivot axis causes a pivoting movement of the top part about the prosthetic knee joint axis.

16. The fastening device according to claim 15, wherein the proximal bearing point is configured to be arranged on the prosthesis socket or on a lever which is pivotable about the socket pivot axis and coupled to the prosthesis socket.

17. The fastening device according to claim 15, further comprising a bracket mounted on the at least one strut and having a receiving device to secure the prosthesis socket to the bracket.

18. The fastening device according to claim 17, wherein the receiving device is part of a pyramid adapter.

19. The fastening device according to claim 17, wherein the bracket is a one-arm or two-arm bracket.

20. The fastening device according to claim 15, wherein the distal bearing point is configured to be arranged on a carrier, the carrier having a coupling device configured to be secured to the top part.
